(19) United States
(12) Patent Application Publication
(10) Pub. No.: US 2021/0321985 A1
(43) Pub. Date: Oct. 21, 2021

(54) COMPOSITIONS AND METHODS FOR TARGETED CONTRAST AGENTS FOR MOLECULAR IMAGING

(71) Applicant: ARIZONA BOARD OF REGENTS ON BEHALF OF THE UNIVERSITY OF ARIZONA, Tucson, AZ (US)

(72) Inventors: Terry Matsunaga, Tucson, AZ (US); Craig C. Weinkauf, Tucson, AZ (US); Evan C. Unger, Tucson, AZ (US); Edmund R. Marinelli, Tucson, AZ (US)

(21) Appl. No.: 17/355,960

(22) Filed: Jun. 23, 2021

Related U.S. Application Data
(63) Continuation of application No. 16/096,940, filed on Oct. 26, 2018, now abandoned.
(60) Provisional application No. 62/329,981, filed on Apr. 29, 2016.

(51) Int. Cl. A61B 8/08; A61B 5/00; A61K 49/22; C07K 16/28; A61B 5/02

(52) U.S. Cl. CPC A61B 8/481; A61B 5/0082; A61B 5/0095; A61K 49/223; B82Y 5/00; A61B 5/02; A61K 49/221; G01N 2800/2871; G01N 2800/50; C07K 16/2836

(57) ABSTRACT

The invention provides novel targeted particles as contrast agents for use in molecular imaging of vulnerable plaque, and methods of preparation and application thereof.

### PRIORITY CLAIMS AND RELATED PATENT APPLICATIONS

This application is a Continuation and claims benefit of U.S. patent application Ser. No. 16/096,940, filed on Oct. 26, 2018, which is a 371 application of PCT/US17/30299, filed on Apr. 29, 2017, which claims priority to Provisional Application No. 62/329,981, filed on Apr. 29, 2016, the content of which is incorporated herein by reference in its entirety.

### TECHNICAL FIELDS OF THE INVENTION

This invention generally relates to compositions of contrast agents for molecular imaging and methods of their preparation and use. More particularly, the invention relates to novel targeted particles as contrast agents for use in molecular imaging of vulnerable plaque, and methods of preparation and application thereof.

### BACKGROUND OF THE INVENTION

Heart attack and stroke are responsible for more deaths and morbidity than any other diseases. Atherosclerosis is a major contributor to coronary heart disease and a primary cause of non-accidental death in Western countries (Coopers, E. S. Circulation 1993, 24, 629-632; WHO-MONICA Project. Circulation 1994, 90, 583-612). The causative element in heart attack and stroke is most often rupture of a vulnerable plaque, which refers to a collection of white blood cells and lipids in the wall of an artery that is particularly unstable and prone to break.

Early and accurate detection and quantitation of vulnerable plaque formation is of major clinical importance as these plaques often progress to stable coronary artery disease or acute ischemic syndromes caused by the rupture of vulnerable plaque.

One current method for locating vulnerable plaque is to peer through the arterial wall with infrared light. A catheter is inserted through the lumen of the artery. The arterial wall is illuminated with infrared light from a delivery fiber. A diffuse reflectance spectroscopy can be used to determine chemical composition of arterial tissue to detect vulnerable plaque.

Another method for vulnerable plaque detection is optical coherence tomography (OCT), which images the arterial tissue surrounding the lumen. A catheter is inserted through the lumen of the artery, which catheter includes a fiber that transports light having a limited coherence length through imaging optics to the arterial wall. The backscattered light couples back into the fiber towards an interferometer. The interferometer provides a cross-correlation signal that is used to map the shape of the arterial tissue. This map of the morphology of the arterial wall is used to detect vulnerable plaque.

In either approach, an invasive procedure cannot be avoided, resulting in added risk to the patient and increased burden to healthcare cost.

There remains an ongoing unmet need for an improved, non-invasive method for detecting vulnerable plaque prior to its rupture allowing timely intervention to prevent heart attack and stroke.

### SUMMARY OF THE INVENTION

The present invention is based in part of the unexpected discovery of a novel molecular probe that enables a non-invasive method for detecting vulnerable plaque prior to its rupture. As disclosed herein, targeted microbubbles are employed as molecular probes to enhance vulnerable plaque detection by ultrasound techniques. The molecular probes of the invention allow accurate imaging, localization and quantification of vulnerable plaque.

More particularly, as disclosed herein, vascular cell adhesion molecule 1 (VCAM-1) is chosen and demonstrated to be a preferred target for vulnerable plaque. Molecular probes (or contrast agents) comprising peptides targeting VCAM-1 can be effectively used to detect vulnerable plaque. The invention is of particular utility for making clinically translatable nanoparticles and microparticles targeted to VCAM-1 for diagnosis and treatment of vulnerable plaque.

In one aspect, the invention generally relates to a molecular probe comprising a microscopic or nanoscopic particle or bubble conjugated thereto a ligand having a binding affinity to VCAM-1 protein.

In another aspect, the invention generally relates to an aqueous emulsion or suspension comprising a molecular probe disclosed herein. In certain embodiments, the emulsion or suspension is in a homogenized form.

In yet another aspect, the invention generally relates to a method for detecting a vulnerable plaque. The method comprises: administering to a subject in need thereof an aqueous emulsion or suspension of a contrast agent comprising a molecular probe disclosed herein; and imaging a part of the subject to detect the presence of vulnerable plaque. The imaging is preferably that of an ultrasound.

In yet another aspect, the invention generally relates to a method for accessing the risk of heart attack and stroke. The method comprises: administering to a subject in need thereof an aqueous emulsion or suspension of a contrast agent comprising a molecular probe disclosed herein; and imaging a part of the subject to access the risk of the subject for having a heart attack and/or stroke. The imaging is preferably that of an ultrasound.

### BRIEF DESCRIPTION OF THE DRAWINGS

The invention will be better understood from a reading of the following detailed description taken in conjunction with the drawings in which like reference designators are used to designate like elements, and in which:

FIG. 1 shows binding of targeted microbubbles to HAECs under flow conditions: The number of microbubbles that bound per human aortic endothelial cell (HAEC) and the shear stress at which these bubbles were binding.

FIG. 2 shows that carotid endarterectomy (CEA) plaques have differential histology features.

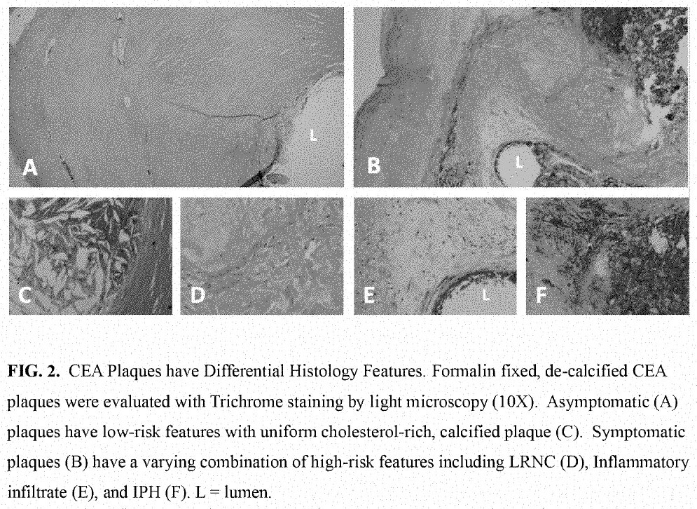

FIG. 3 shows representative images of staining levels and graphical data illustrating the results for the staining of asymptomatic and symptomatic carotid plaques by the anti-VCAM-1 antibody.

FIG. 4 shows exemplary data of left ventricular cavity video signal intensity vs time for targeted microbubbles in double knockout (DKO) mice.

FIG. 5 shows exemplary video intensity of plaque containing region in DKO mice at 5 minutes post injection with targeted microbubbles.

FIG. 6 shows cardiac imaging of wild-type (control) and DKO (atherosclerotic) mice with targeted microbubbles.

FIG. 7 shows an exemplary scheme for production of bioconjugates using an exemplary peptide DYTWFELWDMMO (SEO ID NO:9).

FIG. 8 shows an exemplary embodiment of a structure of a VCAM-1 targeting bioconjugate.

FIG. 9 shows an exemplary synthetic scheme for the production of pure bioconjugate using alkyne-azide cycloaddition.

FIG. 10 shows exemplary preparation of a VCAM-1 binding peptide bioconjugate using the diethylsquarate-conjugation methodology and HGRANLRILARY (SEQ ID NO:10) as the peptide.

### DETAILED DESCRIPTION OF THE INVENTION

The invention provides a novel molecular probe that enables a non-invasive method for detecting vulnerable plaque prior to its rupture. More particularly, targeted microbubbles are employed as molecular probes to enhance detection of vulnerable plaque by ultrasound. The molecular probes of the invention allow accurate imaging, localization and quantification of vulnerable plaque.

As disclosed herein, VCAM-1 is a preferred target as evidenced by in vino binding studies in human endothelial cells, by immunohistochemistry of ex vivo human carotid plaque showing strong correlation with severity of plaque grade, and is also supported by in vivo studies in atherosclerotic mice. VCAM-1 targeted microbubbles are employed to enhance vulnerable plaque detection by ultrasound, as confirmed by antibody studies in human carotid plaque sections and in vivo mouse imaging studies.

In one aspect, the invention generally relates to a molecular probe comprising a microscopic or nanoscopic particle or bubble conjugated thereto a ligand having binding affinity to VCAM-1 protein.

As used herein, “microscopic or nanoscopic” refers to sizes ranging from nanoscopic (from about 1 nm to about 100 nm) to microscopic (from about 100 nm to about 100 μm).

In some embodiments, each microscopic or nanoscopic particle or bubble is conjugated to a plurality of units of the ligand. The ligand surface density, which is independent of the particle size, may be from about 2,000 to about 400,000 (e.g., from about 2,000 to about 200,000, from about 2,000 to about 100,000, from about 2,000 to about 50,000, from about 2,000 to about 20,000, from about 5,000 to about 400,000, from about 10,000 to about 400,000, from about 20,000 to about 400,000, from about 50,000 to about 400,000, from about 100.000 to about 400,000) ligands per square micron.

Any suitable ligands (e.g., peptides, antibodies, minibodies, antibody fragments and scFv may be used) may be used for binding to the target biomarker (e.g., VCAM-1, vWF or p-selectin).

In certain preferred embodiments, the ligand is a peptide. The peptide may have any suitable length, for example, from about 4 to about 20 amino acids in length. In certain embodiments, the peptide has a length of about 4 to about 12 (e.g., 4, 5, 6, 7, 8, 9, 10, 11, 12) amino acids. In certain embodiments, the peptide has a length of about 12 to about 14 (e.g., 12, 13, 14) amino acids. In certain embodiments, the peptide has a length of about 14 to about 20 (e.g., 14, 15, 16, 17, 18, 19, 20) amino acids.

The ligand may be conjugated to the microscopic or nanoscopic particle or bubble via any suitable tether or linker, for example, a polyethylene glycol (PEG) tether of any suitable length. Polyethylene glycol is a polydisperse polymer consisting of a collection of polymers that constitute an envelope distributed at values lower than, equal to and larger than the given average molecular weight. This is well understood by those skilled in the art. Therefore, for the purposes of this discussion, molecular weight is given as the average molecular weight. In certain embodiments, the PEG tether has an average molecular weight ranging from about 1,000 to about 20,000 (e.g., from about 1,000 to about 15,000, from about 1,000 to about 10,000, from about 1,000 to about 5,000, from about 2,000 to about 20,000, from about 5,000 to about 20,000, from about 10,000 to about 20,000, from about 2,000 to about 10,000, from about 5,000 to about 20,000). In certain embodiments, the PEG tether has an average molecular weight about 2,000. In certain embodiments the PEG tether has an average molecular weight of about 3400. In certain embodiments the PEG tether has an average molecular weight of about 5,000. In certain embodiments the PEG tether has an average molecular weight of about 10,000.

The microscopic or nanoscopic particle or bubble may be solid or hollow, e.g., filled by a gas or gaseous precursor. For ultrasound energy, most preferably the microscopic or nanoscopic particles or bubbles are filled with a gas or gas precursor. For magnetic energy, the particles preferably contain an iron oxide material but may also contain other paramagnetic materials. For optical energy, most preferably the particles contain gold nanoparticles. The structure of the microscopic or nanoscopic particle or bubble can be optimized for the energy regime to be employed.

The microscopic or nanoscopic particle or bubble may have any suitable shape and size. For example, the particle or bubble may take the shape of a sphere, a rod, a cube, or an irregular shape, etc. In some embodiments, the microscopic or nanoscopic particle or bubble is a nanoparticle. In some embodiments, the microscopic or nanoscopic particle or bubble is a microparticle. In some embodiment, the microscopic or nanoscopic particle or bubble is a microbubble filled with a gaseous material.

In some embodiments, the microscopic or nanoscopic particle or bubble has a diameter from about 10 nm to about 10 μm (e.g., from about 10 nm to about 5 μm, from about 10 nm to about 1 μm, from about 10 nm to about 500 nm, from about 10 nm to about 100 nm, from about 50 nm to about 10 μm, from about 100 nm to about 10 μm, from about 1 μm to about 10 μm). In some embodiments, the microscopic or nanoscopic particle or bubble has a diameter from about 10 nm to about 100 nm. In some embodiments, the microscopic or nanoscopic particle or bubble has a diameter from about 100 nm to about 1 μm. In some embodiments, the microscopic or nanoscopic particle or bubble has a diameter from about 1 μm to about 10 μm.

In a preferred embodiment, the microscopic or nanoscopic particle or bubble is a microbubble targeted to VCAM-1. The microscopic or nanoscopic particle or bubble bears a plurality of peptides targeted to VCAM-1. These peptides are affixed to the surface of the microscopic or nanoscopic particle or bubble. The molecular probe comprising a microscopic or nanoscopic particle or bubble conjugated thereto a ligand targeted to VCAM-1 is preferably administered intravenously to the subject in need thereof, e.g., a patient.

The energy applied to the subject may comprise ultrasound energy, magnetic energy, radiofrequency energy, microwave energy, optical energy, gamma ray, positron, electron beam or other energy applied into or onto the subject. In a preferred embodiment, the energy applied to the subject is ultrasound energy.

For example with ultrasound energy and imaging, after background microbubbles have cleared, generally within several minutes, signals from the VCAM-1 targeted microbubbles can be used as a read-out to analyze and quantitate the existence and/or level of seriousness of vulnerable plaque so that appropriate therapies can be instituted to avoid stroke and/or heart attack.

In some embodiments, therapeutic ultrasound can also be applied to a subject in need thereof to treat the vulnerable plaque for therapeutic effect.

In certain embodiments, the microbubble is filled with a gaseous material. The gaseous material can be any suitable gas or a mixture of gases. In certain embodiments, the gaseous material comprises a fluorinated gas.

The term “fluorinated gas”, as used herein, refers to hydrofluorocarbons, which contain hydrogen, fluorine and carbons, or to compounds which contain only carbon and fluorine atoms (also known as perfluorocarbons) and also to compounds containing sulfur and fluorine. In the context of the present invention, the term may refer to materials that are comprised of carbon and fluorine or sulfur and fluorine in their molecular structure and are gases at normal temperature and pressure. Examples of fluorinated gases include sulfur hexafluoride, perfluoropropane, perfluorobutane, perfluoropentane and perfluorohexane, or mixtures thereof.

In certain embodiments, the gaseous material further comprises a suitable percentage of non-fluorinated gas or gas mixture, for example, about 2% to about 20% air or nitrogen (e.g., from about 5% to about 20%, from about 10% to about 20%, from about 15% to about 20%, from about 2% to about 15%, from about 2% to about 10%, from about 2% to about 5% of air or nitrogen).

In certain preferred embodiments, the targeting ligands are peptides and are modified, as disclosed herein, for incorporation into or onto the surface of the microscopic or nanoscopic particle or bubble. The peptides may comprise L- and D-amino acids and mixtures thereof. Preferably, the peptides are from about 4 to about 20 amino acids in length (e.g., from about 4 to about 16, from about 4 to about 14, from about 4 to about 12, from about 8 to about 20, from about 10 to about 20, from about 12 to about 20 amino acids). More preferably, the peptides may be from about 12 to about 14 amino acids in length (e.g., 12, 13, or 14 amino acids).

In certain embodiments, antibodies, minibodies, antibody fragments and scFv may be used instead of peptides as targeting ligands.

Targeting ligands also include those modified from the specific peptides, antibodies, minibodies, antibody fragments and scFv disclosed herein bur maintain at least 70%, at least 75%, at least 80%, at least 85%, at least 90%, at least 95%, or at least 98% homology with a peptide sequence disclosed herein.

In certain embodiments, the targeting ligand comprises a sequence of RANLRILARY (SEQ ID NO: 1). In certain embodiments, the targeting ligand comprises a sequence modified from RANLRILARY (SEQ ID NO: 1) but having at least 70%, at least 75%, at least 80%, at least 85%, at least 90%, at least 95%, or at least 98% homology.

The molecular probe may comprise a microscopic or nanoscopic particle or bubble that is conjugated thereto a second (or even a third) ligand having binding affinity to vWF or p-selectin. In certain embodiments, the second (or the third) ligand has binding affinity to vWF. In certain embodiments, the second (or the third) ligand has binding affinity to p-selectin.

In certain embodiments of the molecular probe, the microbubble is coated by a film-forming material. In certain embodiments, the film-forming material may comprise a phospholipid or a mixture of phospholipids. In certain embodiments, the film-forming material comprises lipids that are not phospholipids. In certain embodiments the film-forming material may constitute combinations of phospholipids and lipids that are not phospholipids.

Any suitable lipids may be utilized. The lipid chains of the lipids may vary from about 10 to about 24 (e.g., from about 10 to about 20, from about 10 to about 18, from about 12 to about 20, from about 14 to about 20, from about 16 to about 20, 10, 11, 12, 13, 14, 15, 16, 17, 18, 19, 20, 21, 22, 23, 24) carbons in length. More preferably, the chain lengths are from about 16 to about 18 carbons.

The lipid chains may be saturated or unsaturated but are preferably saturated. Cholesterol and cholesterol derivatives may also be employed with the proviso that they be neutral, or if negatively charged contain a head group greater than about 350 MW in juxtaposition to the negative charge to shield the charge from the biological milieu.

Examples of the film-forming material includes: phosphatidylcholine (PC) which is a phospholipid containing the choline headgroup —O*—CH₂—CH₂—N⁺(CH₃)₃, phosphatidylethanolamine-monomethoxy-polyethyleneglycol (PE-MPEG) which is a phospholipid containing the head group —O*—CH₂CH₂N—C(═O)—O—(CH₂CH₂O)ₙ—CH₃, phosphatidylethanolamine (PE) which is a phospholipid containing the head group —O*—CH₂CH₂—NH₂, and phosphatidylethanolamine-polyethyleneglycol-linker-ligand (PE-PEG-linker-ligand.) which is a phospholipid containing the headgroup O*—CH₂CH₂N—C(═O)—O—(CH₂CH₂O)ₙ—CH₂—CH₂—X-ligand, where X is any functionality or series of groups, that links the targeting ligand to the polyethylene glycol chain.

As used herein, the term “phospholipid” refers to a structure such as R¹—C(═O)—O—CH₂—C(H)—O—(O═C—R²)—CH₂—O—[P(═O), —O*, —O⁻], where the oxygen bearing the asterisk is the same as that shown in the definition of each of the head groups and R¹ and R² are long chain (e.g., from about 10 to about 24 carbons in length) fatty esters which may be saturated or unsaturated singly or in combination and may or may not be of the same length. These definitions exemplify the terms and are well known to those skilled in the art and are described in further detail below. For example, the PE-PEG-ligand bioconjugate, which synonymous with the PE-PEG-linker-ligand, comprises from 0.05 mole percent to about 5 mole percent of total lipid. More preferably, the PE-PEG-ligand bioconjugate comprises from about 0.1 mole percent to about 1 mole percent of total lipid.

While the molecular probe of the invention may preferably be that of a microbubble, the molecule probe of the invention can also be a probe comprising a nanoparticle or microparticle of an iron oxide (or a mixture of iron oxides) or a gold nanoparticle or nanorod.

In another aspect, the invention generally relates to an aqueous emulsion or suspension comprising a molecular probe disclosed herein.

As used herein, an “emulsion” refers to a heterogeneous system consisting of at least one immiscible liquid dispersed in another in the form of droplets that may vary in size from nanometers to microns. The stability of emulsions varies widely and the time for an emulsion to separate can be from seconds to years. Suspensions may consist of a solid particle or liquid droplet in a bulk liquid phase. As an example, an emulsion of dodecafluoropentane can be prepared with phospholipid or fluorosurfactant and the bioconjugate incorporated into the emulsion at a ratio of from about 0.1 mole percent to about 1 mole percent or even as much as 5 mole percent, relative to the surfactant used in stabilizing the emulsion.

In certain embodiments, the emulsion or suspension is in a homogenized form.

As used herein, a “homogenized” form refers to wherein the emulsion or suspension has been prepared with a form of vigorous mixing. Homogenization can be achieved by any of several processes used to make a mixture of two mutually non-soluble liquids the same throughout. This is generally achieved by turning one of the liquids into a state consisting of extremely small particles distributed uniformly throughout the other liquid. Homogenization is typically conducted using instruments, e.g., an ultra turrax type, a ultrasonic probe mixer/homogenizer, or a high pressure homogenizer which forces the constituents of the mixture to be emulsified or suspended by forcing them through a small opening or a valve whose interior size can be adjusted, at high pressure.

In yet another aspect, the invention generally relates to a method for detecting a vulnerable plaque. The method comprises: administering to a subject in need thereof an aqueous emulsion or suspension of a contrast agent comprising a molecular probe disclosed herein; and imaging a part of the subject to detect the presence of vulnerable plaque.

The imaging is preferably that of an ultrasound. For ultrasound imaging, an ultrasound probe may be applied to a body surface and imaging performed using diagnostic ultrasound. Endoscopic ultrasound or intravascular ultrasound may be performed.

For magnetic resonance imaging, the subject. e.g. a patient, is generally placed into a superconducting magnet. Oscillating radiofrequency fields are applied to perturb the magnetization and signals are received.

For optical imaging, light energy is applied, either externally or via a catheter. The light energy may range from light in the visual spectrum, to that in the ultraviolet, to that in the infrared (IR) or near IR spectral region. When light energy strikes the particles, e.g., gold nanoparticles, it may be reflected and received by a sensor. Alternatively, the light energy may interact with the particle to create an ultrasound signal. e.g., photoacoustic response. In this case light, generally from a laser, is used to stimulate the particle and an ultrasonic sensor is used to receive and process the ultrasound signal emitted therefrom.

In yet another aspect, the invention generally relates to a method for accessing the risk of heart attack and stroke. The method comprises: administering to a subject in need thereof an aqueous emulsion or suspension of a contrast agent comprising a molecular probe disclosed herein; and imaging a part of the subject to access the risk of the subject have a heart attack and stroke.

The imaging is preferably that of an ultrasound.

### EXAMPLES

To study which molecular targets would potentially be useful for targeting microscopic or nanoscopic particles or bubbles to vulnerable plaque, four molecular targets were selected and peptides expected to bind to the selected targets were identified. The targets and binding peptides are shown in Table 1.

TABLE 1
Targets and Ligands in Vulnerable Plaque

| | | Evaluated |
|---|---|---|
| | | in Species |
| Target | Ligand | Target Protein |
| VCAM-1 | RANLRILARY (SEQ ID | 1 (human, |
| | NO: 1) | mouse) |
| VCAM-1 | VHPKQHRGGSY (SEQ ID | 2 (mouse) |
| | NO: 2) | |
| VCAM-1 | CNNSKSHTC (SEQ ID | 3 (human, |
| | NO: 3) | mouse) |
| Von | RVVCEYVFGRGAVCSA | 4 (human), |
| Willebrand | (SEQ ID NO: 4) cyclic | 5 (human |
| Factor | (4-14)-disulfide, | and mouse) |
| (vWF) | Recombinant | |
| | human GPIbα (GPIb | |
| | 1-290) | |
| p-Selectin | LVSVLDLEPLDAAWL | 6 (mouse) |
| | (SEQ ID NO: 5) | |
| LOX-1 | LSIPPKA (SEQ ID NO: 6), | 7 (human) |
| | FQTPPQL (SEQ ID NO: 7), | |
| | LTPATAI (SEQ ID NO: 8) | |

Expression of VCAM-1 was verified in a mouse model and was also performed in human specimens of vulnerable plaque. Phospholipid-PEG-linker-peptide conjugates were prepared to allow preparation of clinically translatable microbubbles targeted to the following molecular targets present in vulnerable plaque: VCAM-1, dysregulated VWF multimers, P-selectin and LOX-1.

Microbubbles were prepared and tested in vitro in a flow cell containing HAECs and by immunohistochemistry (IHC) using antibodies to the respective molecular targets on human carotid plaque sections. Both indicated VCAM-1 as the most consistent target. In vivo studies using contrast enhanced ultrasound molecular imaging as a readout in a murine model of advanced inflammatory atherosclerotic plaque also indicated that VCAM-1 was most consistently differentially detectable in inflammatory plaque in vino and in vivo (DKO mice). LOX-1 and vWF were less consistently detected across the range of in vitro and in vivo studies conducted to date.

The studies revealed that VCAM-1 targeted microbubbles enhanced vulnerable plaque detection by ultrasound. This was confirmed by antibody studies in human carotid plaque sections and in vivo mouse imaging studies.

Results of binding of targeted microbubbles to HAECs in a flow chamber are described below. An aspect of this experiment was testing the shear stress at which the microbubbles could still bind avidly. This was conducted with the use of a flow chamber and adjusting flow rates followed by the determination of shear stress. Data was collected that helped determine maximal binding of microbubbles as a function of shear stress. In addition, a decrease in binding from maximum was used to provide insight into the “unbinding” of microbubbles. This was useful for comparing the relative affinity of ligands for the peri-inflammatory region around vulnerable plaque.

In these experiments, five different types of targeted microbubbles were tested as well as one control microbubble that lacked a targeting ligand. The ligands tested were all peptide-based with selective affinity to VCAM-1, VWF, and several different ligands to LOX-1 (in particular, the ligands were designated as LOX-1-6, LOX-1-8, and LOX-1-10). In these experiments, the targeted microbubbles were flowed through a BIO-RAD Econo peristaltic pump at a dilution of 1:1000 (microbubbles:volume of PBS). The solution reached a fibronectin-coated dish that held HAEC. Microbubbles were stained with DiI, which is a red dye with an excitation of 549 nm and emission of 565 nm. HAECs were stained with calcein, which is a green dye with an excitation of 495 nm and emission of 516 nm. Then, quantification of microbubble binding to the HAEC was performed.

The results of the flow chamber microbubble binding studies were normalized with respect to the applied shear stress and appear in Table 2.

TABLE 2
Control and Targeting Microbubble Binding to Human Aortic
Endothelial Cells under Flow Conditions

| | Shear Stress | Microbubbles |
|---|---|---|
| Microbubble | (dynes/cm²) | bound/cell* |
| Control | 4.34 | 0 |
| | 8.68 | 0 |
| | 13.02 | 0 |
| VCAM-1 | 4.34 | 2.6 |
| | 10.85 | 1.86 |
| | 15.19 | 1.54 |
| | 19.53 | 1.11 |
| | 27.78 | 1.06 |
| vWF | 4.34 | 0.59 |
| | 13.02 | 0.27 |
| | 26.04 | 0.09 |
| LOX-1-6 | 4.34 | 0.71 |
| | 10.85 | 0.25 |
| | 15.19 | 0 |
| | 17.36 | 0 |
| | 19.53 | 0.03 |
| LOX-1-8 | 4.34 | 0.60 |
| | 13.02 | 0 |
| LOX-1-10 | 4.34 | 0.5 |
| | 13.02 | 0.17 |

*More microbubbles were being introduced to the HAEC at higher shear stresses. For example, if the flow is increased from 1 mL/min to 2 mL/min, twice the amount of microbubbles is being delivered. To account for this, the total amount of microbubbles bound to HAEC was divided by the flow rate at which they were delivered.

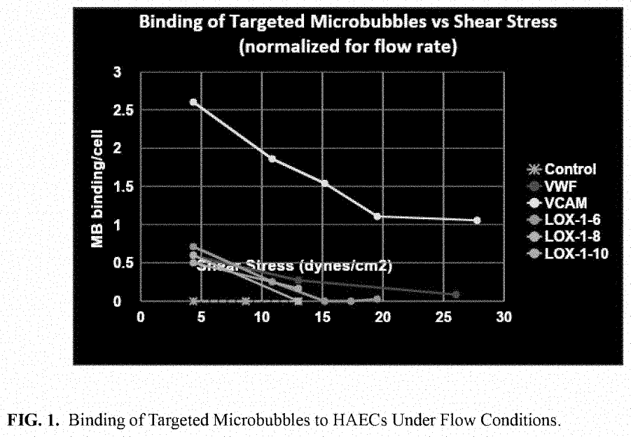

FIG. 1 shows the number of bound microbubbles per HAEC, as well as the shear stress at which these bubbles were binding.

These results show that the targeted microbubbles bound HAEC, while the control microbubbles did not. VCAM-1 targeted microbubbles showed the most binding to HAEC, with about 2.6 microbubbles/cell at a shear stress of 4.34 dynes/cm. When running these experiments at higher shear stresses there were more microbubbles bound to HAEC; however, it was necessary to normalize with respect to shear rate (hence higher flow rate) which presents more microbubbles were to the HAEC. After normalizing the data, it became evident that microbubbles optimally bound to HAEC at lower shear stresses.

Immunohistochemical studies using antibodies to VCAM-1, LOX-1, vWF, and P-selectin were conducted. Antibodies for detection of the molecular targets on human carotid plaques are listed below—Target/Clone/Source: P selectin/RB40.34/Pharmingen; VCAM-1/MK2.7/eBiosciences; LOX-1/23C11 or AF1564/AbCam; VWF/Polyclonal/AbCam. Tissues from 50 human carotid endarterectomy specimens were obtained. Of these, 41 (23 symptomatic and 18 asymptomatic) were evaluable (9 were not evaluable due to poor staining).

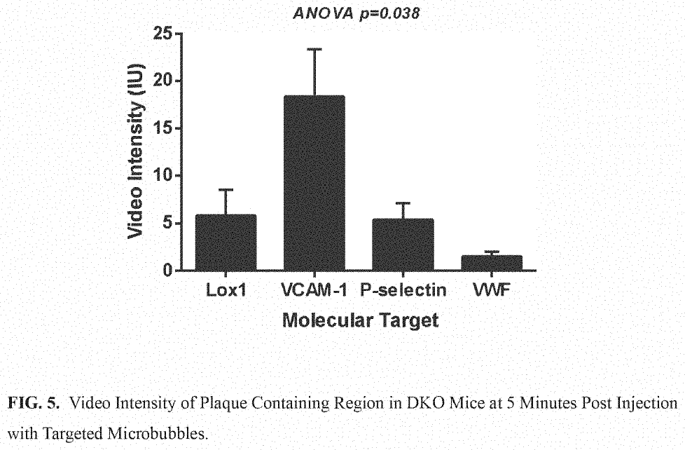

Histological analysis with trichrome staining confirmed that high- vs low-risk plaque features were associated with symptomatic and asymptomatic plaques, respectively (FIG. 5). Formalin fixed, de-calcified CEA plaques were evaluated with Trichrome staining by light microscopy (10×). Asymptomatic (A) plaques have low-risk features with uniform cholesterol-rich, calcified plaque (C). Symptomatic plaques (B) have a varying combination of high-risk features including LRNC (D), Inflammatory infiltrate (E), and IPH (F); L=lumen.

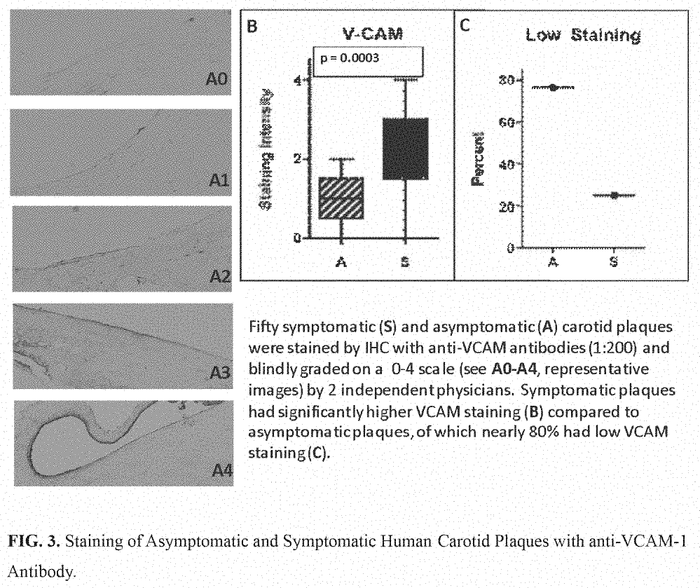

The intensity of staining was evaluated by two independent physicians using a graded system (0-4). Antibodies to LOX-1, vWF and P-selectin did not show significantly different binding to symptomatic vs asymptomatic plaques. The anti-VCAM-1 antibody displayed a strong trend for binding to symptomatic plaques (˜80%) vs asymptomatic plaques (˜20%). Representative images of staining levels and graphical data illustrating the results for the staining of asymptomatic and symptomatic by the anti-VCAM-1 antibody are displayed in FIG. 3. Overall, P-selectin, Lox-1 and VWF showed weak staining on plaques and did not correlate with plaque grade. FIG. 3 shows that immunohistochemistry staining of plaques for VCAM-1 enable differentiation of vulnerable from stable plaque. Thus, ex vivo studies of human carotid endarterectomy specimens showed that staining for VCAM-1 correlated with severity of plaque and degree of symptoms.

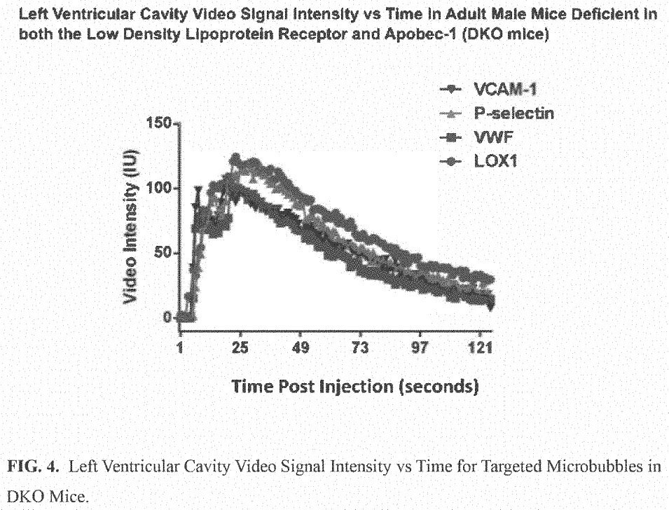

Binding of targeted microbubbles in vivo using a mouse model of vulnerable plaque was studied. Twenty adult male mice deficient in both the low-density lipoprotein receptor and Apobec-1 (DKO mice) were used for in vivo studies. These mice develop age-dependent atherosclerosis and were studied at 40 weeks of age. Eight age-matched wild-type mice were studied as negative controls. All mice received 10 microliters/Kg of each of 4 microbubble formulations: VCAM-1. VWF, P-selectin and LOX-1 targeted microbubble formulations. Microbubble preparations were injected IV via jugular cannula in random order under anesthesia using isoflurane. FIG. 4 shows video intensity measurement of the left ventricle. It was performed to ensure that the relative signal intensity and the duration of circulation was similar between agents as both of these parameters could influence the total signal from adherent microbubbles. In the case where all adhesion of microbubbles was to be non-specific, and if for example, VCAM-1 bubbles recirculated much longer than targeted bubbles, then the number that will eventually adhere would be relatively greater than that found for a similarly adherent targeting microbubble. This was not the case, as demonstrated in by FIG. 4 which shows that the video intensity decay in the bloodstream for the VCAM-1 targeted microbubbles in the left ventricular space was nearly the fastest of the targeted microbubbles tested.

Plaques in mice were imaged as follows. The ascending aorta and arch were imaged in long axis from a right parasternal imaging plane. Contrast enhanced ultrasound (CEU) with each agent were performed 5 min (300 secs, where LV video intensity is near zero IU) after injection. Several frames were obtained with high-power imaging, with mechanical index (MI) of 1.2. Microbubbles in the sector were then fully destroyed by imaging at a MI of 1.9, and several post-destruction frames were obtained at an MI of 1.2 and a pulsing interval of 1 second. A single image reflecting only retained microbubbles was created by digitally subtracting frames from the first pre-destruction frame.

Intensity measurements and pixel intensity threshold analysis were performed from a region of interest placed around the ascending aorta and arch guided by imaging at 14 MHz. Comparisons between agents was made with one-way ANOVA and, when significant (p<0.05), post-hoc analysis with unpaired Students t-test and Bonferroni correction for multiple comparisons was performed. The video intensity at the plaque for the four targeting microbubble formulations is shown in FIG. 5. This data is consistent with the in vitro flow chamber studies which displayed much higher retention of VCAM-1 targeted microbubbles on HAECs in the flow chamber (vide supra) and with the in vitro staining results on human carotid plaques described above in which only anti VCAM-1 antibody differentially stained symptomatic vs asymptomatic plaques.

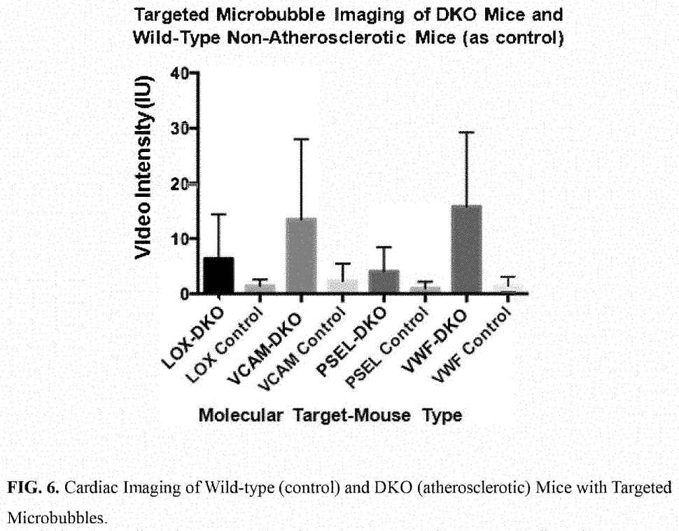

An additional in vivo mouse cardiac imaging study employed microbubbles targeted to the four molecular targets (Lox-1, P-selectin, VCAM-1 and vWF) for imaging of wild type non-atherosclerotic mice and the atherosclerotic DKO mice. In this case, the relative performance of the targeted bubbles was evaluated with respect to the mouse genotype rather than with respect to the microbubble characteristic (i.e., targeted microbubble vs. non-targeting microbubble). Significantly enhanced video intensity in DKO mice vs wild-type mice was observed for VCAM-1, Lox-1 and vWF targeting bubbles as shown in FIG. 6. In this experiment, vWF binding was significant but the results from the human experiments with ex vivo carotid plaque indicated that VCAM-1 was the preferred target.

These studies demonstrated that VCAM-1 is the best target as evidenced by binding studies in in vitro studies in human endothelial cells, by immunohistochemistry of ex vivo human carotid plaque showing strong correlation with severity of plaque grade and is also supported by in vivo studies in atherosclerotic mice.

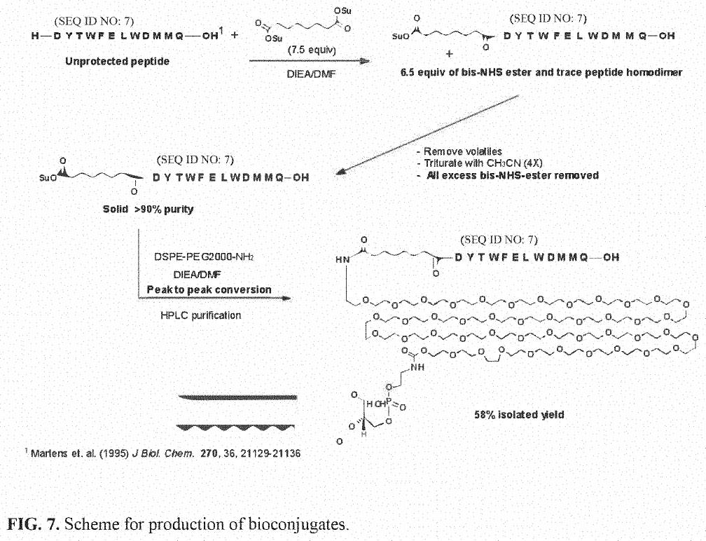

In the case of microbubbles, VCAM-1 targeting peptides were incorporated into bioconjugates for incorporation into microbubbles as described herein. FIG. 7 shows an exemplary scheme for preparation of a phospholipid-PEG2000-peptide conjugate (referred to herein as a bioconjugate) for targeting E-Selectin. This same scheme was employed to couple the above-listed peptides, whose N-terminal amino acid was serially derivatized with the 8-amino-3,6-dioxaoctanoyl linker (ADOA) to phospholipid-PEG2000-amine derivatives such as dipalmitoylphosphatidylcholine-PEG2000-amine and distearoylphosphatidylcholine-PEG2000-amine. Attaching the linker to the N-terminus does not affect binding of the ligands to their targets.

A peptide was dissolved in a suitable solvent such as dry dimethylformamdide (DMF), dimethylacetamide (DMA) or N-methylpyrrolidine (NMP). An excess (about 5-fold to 10-fold) of di-succinimidylsuberate (DSS) was dissolved in one of the solvents specified as for the peptide and stirred at ambient temperature. Then, a large excess (20×-30×) of N,N-diisopropylethylamine was added to the solution of DSS with stirring at ambient temperature. The solution of the peptide was added drop wise over a period of 2 min and the mixture was stirred for 15 min. The volatiles were removed under high vacuum and the resulting residue was triturated several times with a solvent such as ether, ethyl acetate or acetonitrile (if desired the mono-NHS ester of the peptide can be purified by high performance liquid chromatography using gradient elution). The solid residue containing the mono-NHS ester of the suberoylated peptide was then dissolved in dry DMF and dipalmitoylphosphtidylethanolamine-PEG2000-amine (0.9 equiv) dissolved in DMF was added drop wise to the solution of the peptide mono-NHS ester. The mixture was stirred for 16 hr at ambient temperature to give the desired product after HPLC purification and lyophilization of pure product containing fractions. In some cases, it was possible to react the linker-bearing peptide directly with a phospholipid-PEG2000 derivative wherein the terminus of the PEG distal to the phospholipid moiety was functionalized with an NHS ester function.

For production of the targeted microbubbles, one mole percent of the bioconjugate was mixed with 90 mole percent dipalmitoylphosphatidylcholine (DPPC) and 9 mole percent 1,2-dipalmitoyl-sn-glycero-3-phosphoethanolamine-N-[methoxy(polyethylene glycol)-2000] (ammonium salt), or DPPE-MPEG(2000). The lipids were blended and suspended in a diluent of buffered normal saline, propylene glycol and glycerol 85/10/5 (v/v/v). The clear suspension of lipids was placed in a sealed vial containing volatilized dodecaperfluoropentanne (DDFP) gas. The rationale for employing the DDFP is as follows: The FDA approved product, Definity®, which is not a targeting microbubble, contains perfluoropropane gas (Table 3). The lifetime of the microbubble in the vasculature, in large part, is related to the Ostwald partition coefficient (Table 3) of the gas and its molecular weight. For preparation of targeted microbubbles, the gases listed in Table 4 may be selected, although air or nitrogen or other gases can also be employed in admixture with the fluorinated gases.

TABLE 3
Properties of Gases Employed for Preparation of Microbubbles

| | | Ostwald Partition | |
|---|---|---|---|
| Gas | MW | Coefficient²² | Boiling Point |
| Perfluoropropane | 188 | 5.2 × 10⁻⁴ | −36.7 |
| Perfluorobutane | 238 | 2.02 × 10⁻⁴ | −1.7 |
| Perfluoropentane (DDFP) | 288 | 1.17 × 10⁻⁴ | 29 |
| Perfluorohexane | 338 | 2.3 × 10⁻⁵ | 56.6 |

Additional lipids may be incorporated into the microbubble formulation. In a preferred embodiment, the formulation includes a neutral base (bulk shell) lipid such as DPPC at about 82 mole %, a PEG'ylated lipid, e.g., DPPE-MPEG(5000) {1,2-dipalmitoyl-sn-glycero-3-phosphoethanolamine-N-[methoxy(polyethylene glycol)-5000](ammonium salt)} at about 8-10 mole percent, a cone-shaped lipid such as DPPE (dipalmitoylphosphatidylethanolamine) at about 8-10 mole percent and the targeting lipid, e.g., DPPE-PEG(5,000)-linker-peptide at about 1 mole %.

As disclosed herein, lipids of different lipid chain length may be used, for example, from about 10 to about 24 carbons in length. Preferably, the chain lengths are from about 16 to about 18 carbons. The lipid chains may be saturated or unsaturated but are preferably saturated. Cholesterol and cholesterol derivatives may also be employed with the proviso that they be neutral, or if negatively charged contain a head group greater than about 350 MW in juxtaposition to the negative charge to shield the charge from the biological milieu.

The preferred gases for preparation of the microbubbles of the invention are shown in Table 3. Preferred gases include perfluoropropane, perfluorobutane and perfluoropentane. Sulfur hexafluoride may also be used.

In addition to the gas, the other major factor affecting microbubble stability is the composition of the phospholipid in the shell. When one of the inventors (Unger) developed Definity®, it was discovered that gel state lipids are much more effective in stabilizing microbubbles than liquid crystalline state lipids. Definity® is composed of 16 carbon chain length lipid and is in the gel state at physiological temperature.

Increasing the chain length further, e.g., to 18, 19 or 20 carbon length lipid results in a linear increase in bubble stability but decreases membrane elasticity.

TABLE 4
Dissolution Time for a 2.5-micron Encapsulated
Microbubble in Air-saturated Liquid

| Gas | Dissolution time |
|---|---|
| C₃F₈ | 42 min |
| C₄F₁₀ | 83 hours |
| C₅F₁₂ | 17 days |
| C₆F₁₄ | 17 days 14 hours |

As shown in Table 4, microbubbles containing C₅F₁₂ (dodecafluoropentane) gas are about five-times more stable than microbubbles containing C₄F₁₀ (decafluorobutane) gas and nearly 14,000 times more stable than microbubbles containing C₃F₈ (octafluoropropane) gas. Note also that there is only minimal increase in stability of C₆F₁₄ (tetradecafluorohexane) containing microbubbles compared to those containing C₅F₁₂.

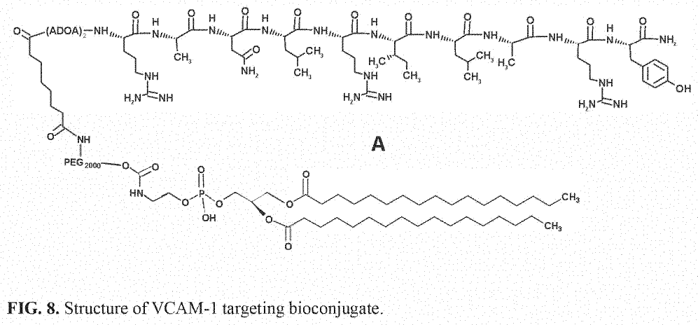

The structure of a VCAM-1 targeted bioconjugate, disclosed herein to be suitable for detecting vulnerable plaque, is shown in FIG. 8. This bioconjugate, when incorporated into microbubbles, gave significantly more signal enhancement than non-targeted microbubbles.

Bioconjugates of the VCAM-1 targeting peptide bearing phospholipid-PEG moieties tethered to the peptide by a linking function are prepared and then incorporated into microbubbles. For example, the phospholipid portion of the conjugate can be selected from phospholipid PEGs as shown in general formula 1.

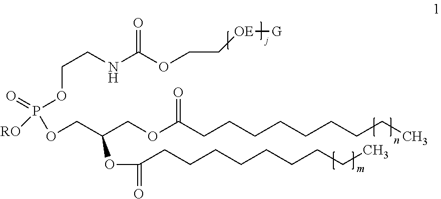

wherein OE represents oxyethylene units (OCH₂CH₂) of a polyethyleneglycol chain, G represents a group that can be employed in a ligation (bond formation) of the phospholipid-PEG assembly to the targeting peptide, and R is a counterion, for example, selected from cationic species such as metal ions or mono-, di-, tri- or tetrasubstituted ammonium ion species, as provided further herein.

In some embodiments, the value of in and n can be equal or different and each independently can be any integer from 0 to 14 (e.g., 0, 1, 2, 3, 4, 5, 6, 7, 8, 9, 10, 11, 12, 13, 14).

In some embodiments, one or both fatty acyl chains optionally may contain zero, one, two or three unsaturated C—C bonds at any position within the chain. The unsaturation sites in the fatty acyl chain or chains may be cis or trans double bonds or triple bonds, singly or in any combination in one chain or both.

In certain preferred embodiments, m and n are equal and can be any integer from 4 to 14. In more preferred embodiments, in and n are equal and can be any integer from 6 to 10 and in even more preferred embodiments, m and n are equal and can be any integer from 6 to 8. In most preferred embodiments, m and n are equal and can be any integer from 6 to 8 and there are no unsaturated C—C bonds.

In some embodiments, the value of J can be any integer from 0 to as much as 500 (e.g., from 0 to about 300, from 0 to about 200, from 0 to about 100, from 0 to about 50, from 0 to about 20, from 10 to about 500, from 50 to about 500, from 100 to about 500), which spans a MW range for the polyethyleneglycol chain of up to about 22,000.

In some embodiments, the value of J can encompass an average PEG average MW of about 400. In some embodiments, the value of J can encompass a PEG average MW of about 600. In some embodiments, J can encompass a PEG average MW of about 2,000. In some embodiments, J can encompass a PEG average MW of about 3400. In some embodiments J can encompass a PEG average MW of about 5,000. In some cases the value of J can encompass a PEG average MW of about 10,000. In a preferred embodiment, the average MW of the PEG is about 2,000. In another preferred embodiment, the average MW of the PEG is about 3,400. In yet another preferred embodiment, the average MW of the PEG may be about 10,000. In a most preferred embodiment, the average MW of the PEG is about 5,000.

The group G may be any group suitable for ligation to the targeting peptide via its C- or N-terminus with the N-terminus being most preferred. Such groups may require of one or more intervening chemistry steps to link to the N-terminus, or another reactive group attached to the N-terminus. Examples of such groups include: amino, hydroxyl, sulfhydryl, carboxy, hydrazinyl, azido, propargyl, homopropargyl, allyl, homoallyl, aldehydo, ketoalkyl or ketoaryl, acyl or alkyl alkynyl, acyl or alkyl alkenyl, oxyimino, thioester, carbonylazido, 2-cyanobenzothiazolyl and 2-mercaptoethylamino. Such groups can react directly with the N-terminus or with another group of suitable reactivity appended to the N-terminus. Such groups may consist of alpha-haloacetyl, carbonyl imidazolyl, carbonyl succinimidyl or other active carbamoyl groups, succinimidylcarbonyl-alkyl-acyl (the second acyl group is in an amide bond at the N-terminus), vinylsulfonyl, alkynylsulfonyl, acrylamido (acrylamides of the N-terminus), propargylamido, acylazido-alkyl-acyl (the second acyl group is appended to the N-terminus via amide linkage), allyl, homoallyl, alkyl or acylalkenyl, aryl aldehyde or aryl ketone where the aromatic ring is appended via another linkage to the N-terminal amino group, a cysteine appended to the N-terminus of the binding peptide, a 2-cyanobenzothiazolyl group tethered to the N-terminus of the peptide which includes or does not include an intervening linker. Note that the 2-cyanobenzothiazolyl moiety reacts in a highly selective manner with peptides which have at their N-terminus a cysteine amino acid. Those skilled in the art understand the suitable pairings of the reactive groups on the PEG and those on the amino terminus of the targeting peptide are employed based on their ability to react with one another efficiently.

Furthermore, the pairings may be reversed in that reactive groups specified for the case of the N-terminus of the peptide can be employed on the PEG and those specified for the PEG could be employed for on the N-terminus of the targeting peptide. The reacting groups given above exemplify those that can be employed to ligate the phospholipid-PEG to the peptide but this does not limit other methods from being applied.

Such methods are contemplated in this application, including for example 4+2 cycloaddition of suitable diene and dienophile partners, and cycloadditions wherein the reacting partners are chosen based on the principle of inverse electron demand.

Further contemplated herein are joining of the appropriate phospholipid PEG with the corresponding N-terminus functionalized peptide using 3+2 cycloaddition reactions such as the reaction of suitable azides or nitrile oxides with alkenyl groups or acylalkenyl groups to provide 1,2,3 triazoles or dihydro-1,2-oxazoles or oxazoles, respectively.

The group R paired with the phospholipid oxygen atom may be selected from inorganic or organic cations. Examples of inorganic cations are Li⁺, Na⁺, K⁺, Cs⁺, Rb⁺ and H⁺ or ammonium. Other singly charged metal ion may also be employed. Examples of organic cations are: monoalkylamino, dialkylamino, trialkylamino, hydroxyalkyl amino, bis-hydroxyalkyl amino, tris-hydroxyalkyl amino, where the alkyl groups are aliphatic (n-alkyl, branched alkyl, cycloalkyl or combinations of these moieties). The alkyl groups may be the same or different.

In some embodiments, the preferred organic cations are: ammonium, trimethylammonium, triethylammonium, diisopropylethylammonium, N-methylmorpholinium, tris-2-hydroxylethylammonium, tris-(hydroxymethyl)methylamino, and N-methylglucammonium (protonated N-methylglucamine), Other trialkylamines, besides those mentioned explicitly herein, wherein the alkyl groups bear hydrophilic substituents, are also contemplated herein.

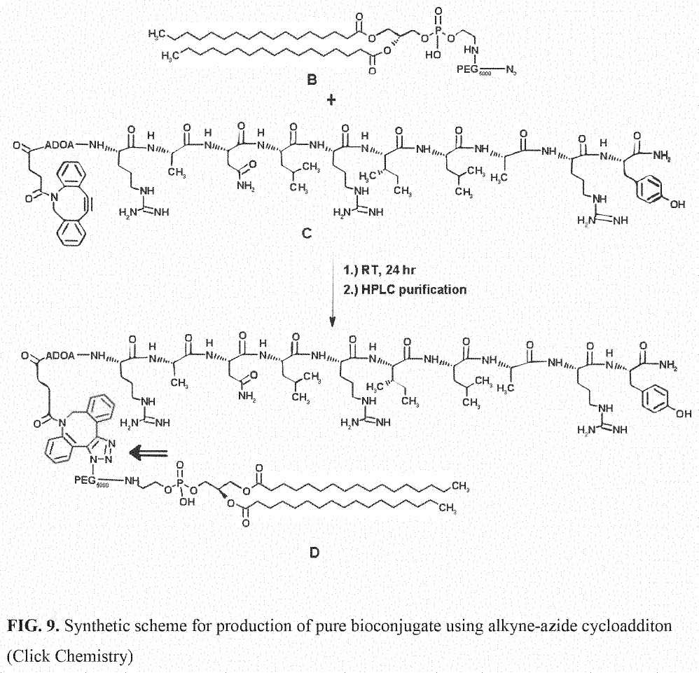

An example of a convenient access to the peptide phospholipid conjugates is shown in FIG. 9. In this case, the phospholipid is dipalmitoylphosphatidyl-PEG2000, which as its azide derivative, was reacted with the peptide bearing an N-terminal dibenzoazacyclooctyne moiety attached to the N-terminus of the peptide.

This approach utilizes highly pure components, which are assembled using strain-promoted copper-free alkyne-azide click (SPAAC) chemistry (FIG. 9). The DSPE-PEG2000-Azide (analog of B) is commercially available from Avanti Polar lipids (the PEG5000 analog B can be obtained via custom synthesis). Compound C, the targeting peptide conjugated to dibenzoazacyclooctyne via the short PEG linker ADOA (8-amino-3,6-dioxaoctanoyl) or a similar linker, was prepared using standard methods of Fmoc solid phase synthesis. Purification by HPLC provided a highly homogeneous material. Incubation of B and C in a mixed aqueous/organic solvent at maximal concentration afforded the desired material via the strain-promoted click reaction of the azido function of B with the cyclooctyne moiety of the N-terminal functionalized peptide C. The reaction resulted in tethering of the input moieties via formation of a tetrazole ring (bold arrow in FIG. 9).

Where any of the moieties on the peptide interfere in the click reaction between the partners, the side chain-protected congener of the targeting peptide C bearing the dibenzoazacyclooctyne moiety can be used. To implement this strategy, cleavage of the side-chain protected peptide-dibenzoazacycloctyne conjugate from an acid sensitive resin such as 2-chlorotrityl or Rink-amide using dilute trifluoroacetic acid in dichloromethane yielded the still side-chain protected peptide. This material was tested for homogeneity first by TLC on silica gel and also by reverse phase or normal phase HPLC analysis and mass spectrometry. A small sample was then subjected to side-chain cleavage with a standard side-chain deprotection reagent such as Reagent B (trifluoroacetic acid/phenol/water/triisopropylsilane (88/5/5/2, v/v/v/v).

Where the resulting side-chain protected congener of peptide C is not of high purity, the on-resin synthesis is optimized or the protected congener of compound C is purified by flash chromatography or HPLC (reverse or normal phase). Having obtained pure side-chain protected compound C it is conjugated to compound B by the click reaction. Treatment of the resulting product using a nonaqueous side-chain cleavage reagent, for example, Reagent P⁺ (trifluoroacetic acid/phenol/methanesulfonic acid (95/2.5/2.5, v/v/v) or Reagent R (trifluoroacetic acid/thioanisole/ethanedithiol/anisole (90/5/3/2, v/v/v/v) to remove the peptide protecting groups, followed by neutralization of the mixture with a mild organic base such as triethylamine or pyridine provided the desired product D without cleavage of the phospholipid fatty esters. Finally, HPLC purification afforded a high purity product.

An alternative to the above described combination of reacting partners is to use the azide-functionalized peptide and the dibenzoazacyclooctyne-functionalized PEG5000-phospholipid system, i.e., where the reactive groups on the reacting partners B and C are ‘swapped’. The same strategies discussed as alternatives in cases where there is interference of the peptide with the click reaction or other incompatibility of reacting partners with the click reaction, discussed above, can be applied for the case of the reaction of the components wherein the reactive moieties are swapped between the phospholipid and the peptide.

Where phospholipid fatty esters cannot be retained in the strategies described above, then a di-ether analog of compound B such as bis-hexadecylphosphatidyl-ethylcarbamoyl-PEG5000-azide where the fatty ester groups are replaced by long chain ether functions is used in lieu of the phospholipid component. This obviates the problem of the acid lability of the phospholipid fatty esters to acid conditions. The diether-phosphatidyl-PEG5000-azide is easily prepared from commercially available bis-hexadecylphosphatidyl-ethanolamine (Bachem Co.) employing standard chemistry well known in the field of functionalized PEGs and phospholipids.

Where the rate of the click reaction is insufficient, the strategies discussed above are employed using more reactive cyclooctyne moieties such as those based on bicyclo[6.1.0]non-4-yn-9-ylmethyl carbonyl moiety.

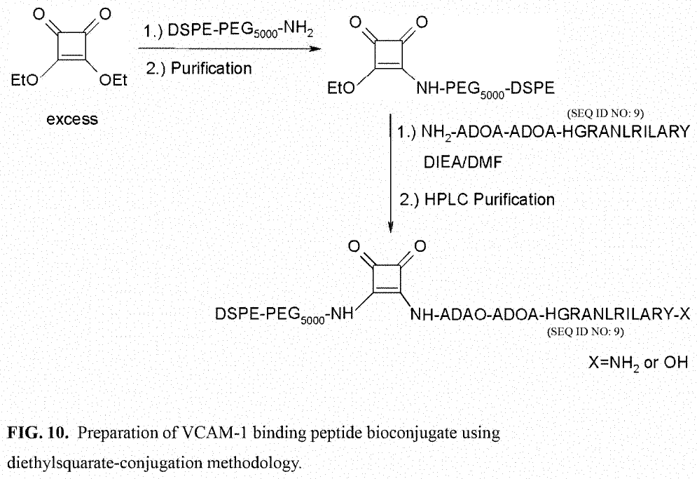

Yet another approach may be employed (FIG. 10). Reaction of bis-hexadecanoyllphosphatidylethylcarbamoyl-PEG5000-amine with diethylsquarate followed by HPLC purification gives a monoadduct, which then can be reacted with the linker functionalized-targeting peptide or its peptide side-chain protected congener. If desired the phospholipid-PEG5000-squarate-linked side-chain protected peptide can be purified by HPLC or flash chromatography. In the case where the side-chains of the peptide are present, treatment of the bis hexadecanoylphosphatidyl-PEG5000 amine-squarate-protected peptide with a nonaqueous side-chain removal reagent such as Reagent P+ or Reagent R removes the peptide protecting groups after which the mixture is neutralized using a mild organic base such as pyridine or triethylamine. Then the volatiles are removed and the final product is purified by HPLC.

Where the VCAM-1 protected peptide is employed, wherein the side chains are not protected, directly after the conjugation reaction with the DPPE-PEG5000-squarate monoadduct the product is purified using HPLC.

In the case where a diether phospholipid PEG5000-amine is employed as the phospholipid component the diether phospholipid PEG5000-amine-squarate-linker-VCAM-1 targeted peptide bioconjugate is purified by HPLC after the conjugation reaction.

Besides the VCAM-1 targeting peptide RANLRILARY (SEQ ID NO: 1) the VCAM-1 targeting peptide CNNSKSHTC (SEQ ID NO: 3) cyclic (1,9)-disulfide that binds to human and primate activated endothelial cells can be employed to prepare microbubbles targeted to VCAM-1 using the described methods.

The bulk shell phospholipid composition comprises octafluoropropane encapsulated in an outer lipid shell consisting of (R)-4-hydroxy-N,N,N-trimethyl-10-oxo-7-[(1-oxohexadecyl)oxy]-3,4,9-trioxa-4-phosphapentacosan-1-aminium, 4-oxide, inner salt, i.e., DPPC, and (R)-∂-[6-hydroxy-6-oxido-9-[(1-oxohexadecyl)oxy]5,7,11-trioxa-2-aza-6-phosphahexacos-1-yl]-ω-methoxypoly(ox-1,2-ethanediyl), monosodium salt, i.e., DPPE-PEG5000 and DPPE. DPPE-PEG5000 has an approximate molecular weight of 5750 Daltons.

Each mL of the clear liquid contains 0.75 mg lipid blend (having 0.046 mg DPPE, 0.400 mg DPPC, 0.304 mg MPEG5000-DPPE and approximately 0.041 mg of the DPPE-PEG5000-linker-VCAM-1-targeting peptide bioconjugate), 103.5 mg propylene glycol, 126.2 mg glycerin, 2.34 mg sodium phosphate monobasic monohydrate, 2.16 mg sodium phosphate dibasic heptahydrate, and 4.87 mg sodium chloride in Water for Injection. The pH is between 6.2-6.8. Note that the linker in the bioconjugate encompasses both the ADOA-ADOA linker and the reacting partners used to ligate the peptide to the DPPE-PEG5000 moiety.

After activation, each mL of the phospholipid-coated microspheres encapsulating a fluorocarbon gas comprises a milky white suspension having a maximum of 1.2×10¹⁰ lipid-coated microspheres, and about 150 μL/mL (1.1 mg/mL) octafluoropropane. The microsphere particle size parameters are listed below:

| | |
|---|---|
| Mean Particle Size | 1.1-3.3 μm |
| Particles Less than 10 μm | 98% |
| Maximum Diameter | 20 μm |

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

Sequence listings and related materials in the ASCII text file named “UNIA_21_18_CON_Sequence_Listing_ST25.txt” and created on Dec. 1, 2020 with a size of about 2 kilobytes, is hereby incorporated by reference.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A molecular probe comprising a microscopic or nanoscopic particle or bubble conjugated thereto a ligand having binding affinity to VCAM-1 protein, wherein the ligand is a peptide ranging from about 4 to about 20 amino acids in length conjugated to the microscopic or nanoscopic particle or bubble via a PEG tether.


SEQUENCE LISTING (10 sequences)

SEQ ID NO: 1 - protein, 10 aa, Artificial Sequence
  VCAM-1 targeting peptide
RANLRILARY

SEQ ID NO: 2 - protein, 11 aa, Artificial Sequence
  VCAM-1 targeting peptide
VHPKQHRGGSY

SEQ ID NO: 3 - protein, 9 aa, Artificial Sequence
  VCAM-1 targeting peptide
CNNSKSHTC

SEQ ID NO: 4 - protein, 16 aa, Artificial Sequence
  Von Willebrand Factor (vWF) targeting peptide
RVVCEYVFGRGAVCSA

SEQ ID NO: 5 - protein, 15 aa, Artificial Sequence
  p-Selectin targeting peptide
LVSVLDLEPLDAAWL

SEQ ID NO: 6 - protein, 7 aa, Artificial Sequence
  LOX-1 targeting peptide
LSIPPKA

SEQ ID NO: 7 - protein, 7 aa, Artificial Sequence
  LOX-1 targeting peptide
FQTPPQL

SEQ ID NO: 8 - protein, 7 aa, Artificial Sequence
  LOX-1 targeting peptide
LTPATAI

SEQ ID NO: 9 - protein, 12 aa, Artificial Sequence
  E-Selectin targeting peptide
DYTWFELWDMMQ

SEQ ID NO: 10 - protein, 12 aa, Artificial Sequence
  VCAM-1 targeting peptide
HGRANLRILARY